(19) United States
(12) Patent Application Publication
(10) Pub. No.: US 2006/0085057 A1
(43) Pub. Date: Apr. 20, 2006

(54) Delivery guide member based stent anti-jumping technologies

(75) Inventors: William R. George, Santa Cruz, CA (US); Frank P. Becking, Palo Alto, CA (US)

Correspondence Address: BOZICEVIC, FIELD & FRANCIS LLP (CARDIOMIND), 1900 UNIVERSITY AVENUE, SUITE 200, EAST PALO ALTO, CA, 94303, US

(73) Assignee: CardioMind

(21) Appl. No.: 10/967,079

(22) Filed: Oct. 14, 2004

(51) Int. Cl. A61F 2/06

(52) U.S. Cl. 623/1.11

(57) ABSTRACT

Medical device and methods for delivery or implantation of prostheses within hollow body organs and vessels or other luminal anatomy are disclosed. The subject technologies may be used in the treatment of atherosclerosis in stenting procedures or a variety of other procedures.

### FIELD OF THE INVENTION

The present invention relates generally to medical devices and methods. More particularly, it relates to delivery systems for implanting prostheses within hollow body organs and vessels or other luminal anatomy.

### BACKGROUND OF THE INVENTION

Implants such as stents and occlusive coils have been used in patients for a wide variety of reasons. One of the most common “stenting” procedures is carried out in connection with the treatment of atherosclerosis, a disease which results in a narrowing and stenosis of body lumens, such as the coronary arteries. At the site of the narrowing (i.e., the site of a lesion) a balloon is typically dilatated in an angioplasty procedure to open the vessel. A stent is set in apposition to the interior surface of the lumen in order to help maintain an open passageway. This result may be effected by means of scaffolding support alone or by virtue of the presence of one or more drugs carried by the stent aiding in the prevention of restenosis.

Various stent designs have been developed and used clinically, but self-expandable and balloon-expandable stent systems and their related deployment techniques are now predominant. Examples of self-expandable stents currently in use are the Magic WALLSTENT® stents and Radius stents (Boston Scientific). A commonly used balloon-expandable stent is the Cypher® stent (Cordis Corporation). Additional self-expanding stent background is presented in: “An Overview of Superelastic Stent Design,” Min. Invas Ther & Allied Technol 2002: 9(3/4) 235-246, “A Survey of Stent Designs,” Min. Invas Ther & Allied Technol 2002: 11(4) 137-147, and “Coronary Artery Stents: Design and Biologic Considerations,” Cardiology Special Edition, 2003: 9(2) 9-14, “Clinical and Angiographic Efficacy of a Self-Expanding Stent” Am Heart J 2003: 145(5) 868-874.

Because self-expanding prosthetic devices need not be set over a balloon (as with balloon-expandable designs), self-expanding stent delivery systems can be designed to a relatively smaller outer diameter than their balloon-expandable counterparts. As such, self-expanding stents may be better suited to reach the smallest vasculature or achieve access in more difficult cases.

To realize such benefits, however, there continues to be a need in developing improved delivery systems. Problems encountered with known systems include drawbacks ranging from failure to provide means to enable precise placement of the subject prosthetic, to a lack of space efficiency in delivery system design. Poor placement, such as by stents “jumping” forward upon deployment, hampers stent efficacy. Space inefficiency in system design prohibits scaling the systems to sizes as small as necessary to enable difficult access or small-vessel procedures (i.e., in tortuous vasculature or vessels having a diameter less than 3 mm, even less than 2 mm).

A system described in U.S. Pat. No. 6,623,518 describes a system in which stent end features are captured by complimentary delivery-device features until released. Due to the space required for producing such structure, the system lacks the space efficiency required to scale down the delivery system to as small of sizes as can be attained with systems according to the present invention.

U.S. Pat. No. 5,733,325 describes a similar approach to preventing axial movement of a stent-graft from migrating upon withdrawal of an overlying sheath. However, instead of interlocking stent-delivery device features, turns/bends at the strut ends are simply captured by spokes attached to a central hub.

Another system for limiting stent movement upon deployment is described in U.S. Pat. No. 6,582,460. This patent discloses spring arms that underlie and interface with a stent upon expansion to prevent axial stent movement upon withdrawal of a sheath. These features occupy extremely valuable space in a delivery system. Indeed, by adding another layer of structure along the body of the stent, the extent to which the system can be miniaturized is limited.

Yet another means of avoiding premature stent release and unwanted axial movement of thereof are presented in U.S. Pat. Nos. 4,768,507; 5,026,377; 5,484,444; 5,702,418; 5,824,041; 6,126,685; 6,302,893; 6,067,551 and 6,669,274. These patent all involve engaging a stent from the inside to control its positioning relative the stent delivery system—and, at lease to some degree, release. These systems add components, bulk and/or system complexity.

In view of the above illustrative examples, it can be appreciated that there exists a need for means of controlling the action of self-expanding stent delivery. In addition, improvement to known systems in order to offer more space efficient solutions would be desirable—especially to enable producing the smallest size delivery systems that are able to access the most difficult anatomy. Accordingly, the present invention may be especially useful in the context of small-vessel or other body lumen applications where very little space in the delivery system, especially in high-expansion ratio stents, exists for such features. Yet, aspects of the present invention may be useful in a variety of settings for reason of their generally applicable effectiveness, potential lower cost of production, ease of use or other reasons as may be appreciated by those with skill in the art upon review of the subject disclosure.

### SUMMARY OF THE INVENTION

The present invention offers a number of stent and stent delivery system designs amendable for use in small vessel (or other hollow body region) applications. The stents incorporated in the subject systems are typically self-expanding upon release from a restraint. In particular, the present invention provides stent delivery systems and methods for delivering self-expanding stents that address the problems with stent “jumping” as noted above.

Together, the stent and a delivery guide provide a stent delivery system. When loaded, the stent is held by the delivery guide member in a collapsed configuration with a tubular sheath or distal restraint. The precise nature of the sheath or restraint used in the system to hold the stent for delivery is not critical to the present invention. So long as the distal end of the tubular member is configured as described below, exemplary actuation approaches and configurations therefore are described in commonly assigned U.S. patent application Ser. Nos. 10/792,657, 10/792,679 and 10/792,684, filed on Mar. 2, 2004 or PCT Application No. US 2004/00008909 filed March 20, 2004, each application being incorporated by reference herein in its entirety.

The present invention concerns various related approaches in avoiding stent jumping upon deployment. These approaches stem from the understanding the inventors hereof have developed regarding the root cause of the effect.

To explain why stents “jump” one must consider the context in which it is observed. Specifically, stent jumping is observed in connection with the deployment of self-expanding stents. Such action occurs when struts or other structure defining a plurality of proximal portions of the stent assume an arrangement during delivery that produces a force vector having a forward-directed component.

The present invention minimizes the production of such forces in connection with stent delivery by releasing the proximal stent strut or leg/arm ends or terminal points in a manner that decreases the spring energy stored in the stent so that upon final release it will not jump forward (appreciably or at all). Stated otherwise, rather than simply attempting to hold onto the ends of a stent until a desired release point, the subject invention seeks to controllably release the stored energy in the end struts (or arms/legs) of a stent that could otherwise contribute to stent jumping.

The manner of controllable release is such that the stent struts or terminal legs are released in a step-wise fashion (e.g., one after another/sequentially, in multiples, etc.). This approach may be implemented irrespective of the particular stent design. In other words, special stent strut end configurations are not required. Furthermore, there is no need to configure the stent strut ends to receive a pin or sprocket member between adjacent struts.

As such, stents selected for delivery guides according to the present invention may be relatively less complex in design. Likewise, they may be of the smallest or most compact/compactable sort.

Together with the stents, the subject delivery guides offer systems according to the present invention providing functionality and an ability to scale to sizes not previously achieved. Consequently, the systems may be used in lieu of a guidewire, such as in a “guidewireless” delivery approach. Still further, rather than providing an “over-the-wire” delivery system in which there is provided a guidewire lumen, variations of the present systems may be provided as “on-the-wire” delivery systems in which the stent is carried by a delivery guide occupying a catheter lumen that would commonly otherwise be used to accommodate a guidewire. Of course, this same lumen may first be used for guidewire passage, followed by exchange for the delivery system guide member.

Whether used in such a manner or otherwise (such as by configuring the subject systems for treating larger peripheral vessels), the present invention includes systems comprising any combination of the features described herein. Methodology described in association with the devices disclosed also forms part of the invention. Such methodology may include that associated with completing an angioplasty, bridging an aneurysm, deploying radially-expandable anchors for pacing leads or an embolic filter, or placement of a prosthesis within neurovasculature, an organ selected from the kidney and liver, within reproductive anatomy such as selected vasdeferens and fallopian tubes or other applications.

More specifically, the subject design for the delivery guide of the invention is especially useful in connection with delivering stents having symmetrically designed struts with respect to cell and/or strut geometry. Utilizing such a stent may be highly advantageous for achieving maximum stent compression and/or providing symmetric loading or interface with opposing anatomy once the stent is emplaced.

The subject stent delivery system comprises a self-expanding stent having a plurality of proximal strut ends and a delivery guide, where the delivery guide comprises a tubular member restraining the stent in a collapsed configuration in which the tubular member is adapted to release the proximal strut ends in a step-wise fashion. Generally, this step-wise release methodology is accomplished by way of the tubular member having a distal opening that varies in its axial extent. This end is arranged with respect to the stent in order to release at least some of a plurality of proximal strut ends in a staged or sequential fashion.

The manner in which the axial extent of the tubes end differs can vary. For instance, instead of having a circular opening that is perpendicular to the axis of the delivery system, the axial extent may vary by way of defining an opening cut along a plane that is canted or offset with respect the an axis of the delivery guide/stent. Such an approach yields a restraint/sheath end having an elliptical cross-section. Other distal-end opening configurations for the tubular member include a zig-zag, multiple facet, and a jogged or stepped cut pattern to provide the varying axial extent.

Still further, the varied or varying axial extent may be provided by way of slits that produce different size flaps. Conceivably, the “slits” may simply be scored or perforated regions that break open to allow corresponding flaps to open-up differentially upon the stent strut ends encountering the same. Other manners of varying the axial extent of the tubular member for restraining the stent may be utilized as well.

By virtue of the tubular member end configuration selected, stent jumping is either lessened relative to other systems or may be altogether (or at least effectively) eliminated. A preferred embodiment of the invention is configured to effect staged release of stent struts such that less than half of the proximal strut ends are left for simultaneous release in finally deploying the stent. More preferably, between about one-third and one-quarter, or as little as one remaining proximal stent strut end is held or constrained between the delivery guide inner member and outer tubular member prior to ultimate stent deployment. In any case, no special stent configuration (beyond having at least a proximal end with at least some individual strut end or termination points) will be required to effect such methodology.

Where an inner or core member is provided, it will be possible to prevent the stent from popping out of the delivery guide prematurely upon release of a number of struts, even where prior strut release occurs in an asymmetrical manner. Without such a core member, however, the subject invention can be effectively practiced especially where the restraint is configured to release proximal stent struts ends with at least some symmetry (e.g., opposite pairs, trios, etc. of struts are released simultaneously).

To effect such action, the end of the tubular member may have a sinusoidal perimeter (as viewed when “unrolled”), or have a “W” or zig-zag shape to offer two stages of strut deployment through its varying axial extent. Alternatively, additional stages of proximal strut deployment may be provided by more complicated tubular member end shapes in effecting step-wise or staged strut end release.

In addition, it will be possible to effect additional multiple stage deployment approaches by further incorporating multi-length stent struts (or at least proximal stent strut ends terminating at different axially-oriented points) in the stent used in the delivery system. Especially where radiopaque strut end features are desired for the stent (such as by welding tantalum pieces thereto or inserting plugs into enlarged strut ends) this coordinated approach may offer further utility, without undue compromise in the stent or delivery system design.

Even when the addition of radiopaque features is not the goal, the asymmetric or staged release-adapted sheath or restraint offered by the present invention can improve existing systems. It can be applied thereto as an improvement not heretofore contemplated or fairly suggested in that no other party has appreciated the possibility of controlling stent jumping by virtue of selective release of stored energy in the stent.

As noted above, the tubular member (be it a simple sheath or an end restraint) as well as its construction (e.g., as in terms of providing a multi-piece or composite structure) may vary. Likewise, the tubular member may be perforated, comprise open windings, or otherwise include open sections. The tubular member may comprise hypotubing, polymeric tubing, braided wire, etc. What is important is that the tubular member provides an elongate hollow body that surrounds an outer diameter or periphery of the prosthetic employed and has a varying axial extent.

By varying the axial extent of the tubular stent restraining member, the anti-jump features are provided in the delivery guide—either exclusively or with additional stent feature contribution. In either case, the system offers elegance in design and cost-effective construction without special interlocking features offering manufacture, loading or other challenges.

As for the deployment methodology in delivering a stent, first a stent delivery system is provided having a tubular member holding a self-expanding stent in a collapsed configuration. Sometimes, the stent will be held upon or over an inner member. Next, the stent is positioned at a target site. Ultimately, the stent is released by withdrawing the tubular member to release at least some of a plurality of proximal stent termination points (e.g., strut ends) in a step-wise fashion to alleviate jumping.

The stepwise end release may be such that at least some of the proximal strut ends are individually released (i.e., released one after another). In connection with certain variations of the invention, it may be the case that all of the proximal strut ends are individually released. Still further, the system may be configured so that adjacent ones of the proximal strut ends are released sequentially. Such a system would be advantageous in that the strut release gently occurs as they “peel” open around the periphery of the prosthesis.

Alternatively, it may be desired that at least some of the proximal strut ends are released in a symmetrical fashion (e.g., in sets of opposite pairs, threesomes, etc.). These groups of struts may be the first struts released or be those remaining and thus, reserved for release after one or more pair, etc. has been released.

In any case, it may be desirable to have struts on opposite sides of a body of the stent be released at the same time to provide a measure of symmetry in deployment. By releasing opposing pairs (or other symmetrically arranged multiples, i.e., trios, etc.) simultaneously or by releasing members of such pairs etc. sequentially, concentric or near concentric arrangement of the stent with respect to the delivery guide member can be achieved throughout deployment.

However the staged or sequential release regimen is accomplished, it may be desired that the delivery device release as small a number of the proximal stent struts as one at the end of deployment. Thus configured, there will be no opposing member force to drive stent jumping. Other approaches minimizing opposing force members are also desirable.

Regardless, the purpose of configuring the delivery system to effect any of the referenced action is so that a large number of struts do not deploy simultaneously with adequate force to cause the stent to jump forward. Yet, even when a significant number of opposing struts are released simultaneously but an adequate number of struts are released prior to this event, stent jumping will be decreased relative to a system in which all of the strut legs are released simultaneously.

### Definitions

The term “stent” as used herein refers to any coronary artery stent, other vascular prosthesis, or other radially expanding or expandable prosthesis or scaffold-type implant suitable for the noted treatments or otherwise. Exemplary structures include wire mesh or lattice patterns and coils, though others may be employed in the present invention.

A “self expanding” stent is a scaffold-type structure (serving any of a number of purposes) that expands by its own action from a reduced-diameter configuration to an increased-diameter configuration. The “diameter” need not be circular—it may be of any open configuration. Self-expanding materials may be so by virtue of simple elastic behavior, superelastic behavior, a shape memory effect (i.e., heat-activated transformation from martinsite to austenite) or some other manner. Since the stents will remain in the subject's body, the material should be biocompatible or at least be amenable to biocompatible coating. As such, suitable self expanding stent materials for use in the subject invention include Nickel-Titanium (i.e., NiTi) alloy (e.g., NITINOL) and various other alloys or polymers.

A “wire” as used herein generally comprises a common metallic member. However, the wire may be coated or covered by a polymeric material (e.g., with a lubricious material such as TEFLON®—i.e. PTFE or PolyTetraFluoroEthylene) or otherwise. Still further, the “wire” may be a hybrid structure with metal and a polymeric material (e.g. Vectra™, Spectra™, Nylon, etc.) or composite material (e.g., carbon fiber in a polymer matrix). The wire may be a filament, bundle of filaments, cable, ribbon or in some other form. It is generally not hollow.

A “core” wire as referred to herein is a member internal to an outer member, such as a tubular member. As a core wire, the member fills or at least substantially fills all of the interior space of the tubular member.

An “inner member” as disclosed herein may be a core member or core wire or be otherwise configured.

A “hypotube” or “hypotubing” as referred to herein means small diameter tubing in the size range discussed below, generally with a thin wall. The hypotube may specifically be hypodermic needle tubing. Alternatively, it may be wound or braided cable tubing, such as provided by Asahi Intec Co., Ltd or otherwise. As with the “wire” discussed above, the material defining the hypotube may be metallic, polymeric or a hybrid of metallic and polymeric or composite material.

An “atraumatic tip” may comprise a plurality of spring coils attached to a tapered wire section. At a distal end the coils typically terminate with a bulb or ball that is often made of solder. In such a construction, the coils and/or solder is often platinum alloy or another radiopaque material. The coils may also be platinum, or be of another material. In the present invention, the wire section to which the coils are attached may be tapered, but need not be tapered. In addition, alternate structures are possible. For instance, molding or dip-coating with a polymer may be employed. In one example, the atraumatic tip may comprise a molded tantalum-loaded 35 durometer Pebax™ tip. However constructed, the atraumatic tip may be straight or curved, the latter configuration possibly assisting in directing or steering the delivery guide to a desired intravascular location.

To “connect” or to have or make a “connection” between parts refers to fusing, bonding, welding (by resistance, laser, chemically, ultrasonically, etc), gluing, pinning, crimping, clamping or otherwise mechanically or physically joining, attaching or holding components together (permanently or temporarily).

### BRIEF DESCRIPTION OF THE DRAWINGS

Each of the figures diagrammatically illustrates aspects of the invention. Of these:

FIG. 1 shows a heart in which its vessels may be the subject of one or more angioplasty and stenting procedures;

FIG. 2A shows an expanded stent cut pattern as may be used in producing a stent according to a first aspect of the invention; FIG. 2B shows a stent cut pattern for a second stent produced according to another aspect of the present invention;

FIG. 3A shows an expanded stent cut pattern as may be used in producing a stent according to a first aspect of the invention; FIG. 3B shows a stent cut pattern for a second stent produced according to another aspect of the present invention;

FIGS. 4A-4L illustrate stent deployment methodology to be carried out with the subject delivery guide member;

FIG. 5 provides an overview of a delivery system incorporating at least one of the subject stents;

FIG. 6 is a side sectional view illustrating the manner in which stent jumping occurs in connection with a simple sheath and pusher system as known in the art;

FIG. 7 is a side sectional view illustrating a second embodiment of the invention in which stent jumping is alleviated by staged release of the proximal strut ends of a stent;

FIGS. 8A and 8B are side and top views, respectively, of a tubular stent-restraining member as shown in FIG. 7;

FIGS. 9A and 9B are side and top views, respectively, of an alternate tubular stent-restraining member similar to that shown in FIG. 7; and

FIGS. 10-13 show expanded cut patterns as may be used in producing tubular stent-restraining members according to an aspect of the present invention.

In the figures, like elements in some cases are indicated by a related numbering scheme. Furthermore, variation of the invention from the embodiments pictured is, of course, contemplated.

### DETAILED DESCRIPTION OF THE INVENTION

Before the present invention is described in detail, it is to be understood that this invention is not limited to particular variations set forth and may, of course, vary. Various changes may be made to the invention described and equivalents may be substituted without departing from the true spirit and scope of the invention. In addition, many modifications may be made to adapt a particular situation, material, composition of matter, process, process act(s) or step(s), to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “and,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs.

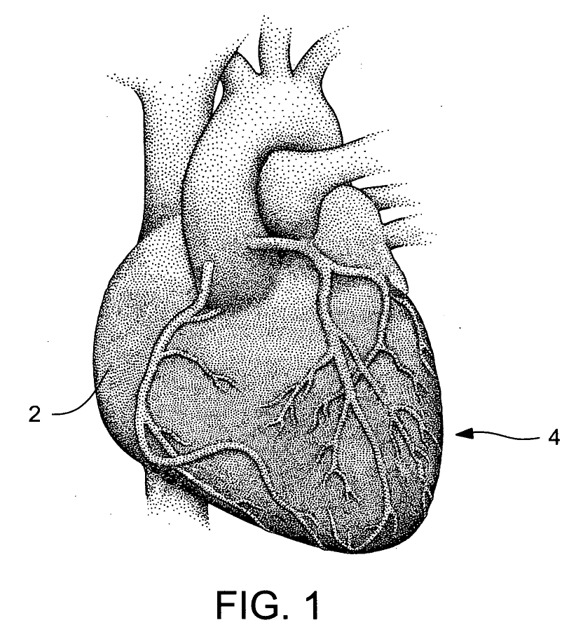

Turning now to FIG. 1, it shows a heart 2 in which its vessels may be the subject of one or more angioplasty and/or stenting procedures. To date, however, significant difficulty or impossibility is confronted in reaching smaller coronary arteries 4. If a stent and a delivery system could be provided for accessing such small vessels and other difficult anatomy, an additional 20 to 25% of coronary percutaneous procedures could be performed with such a system. Such a potential offers opportunity for huge gains in human healthcare and a concomitant market opportunity in the realm of roughly $1 billion U.S. dollars—with the further benefit of avoiding loss of income and productivity of those treated.

Features of the present invention are uniquely suited for a system able to reach small vessels (though use of the subject systems not limited to such a setting.) By “small” coronary vessels, it is meant vessels having an inside diameter between about 1.5 or 2 and about 3 mm in diameter. These vessels include, but are not limited to, the Posterior Descending Artery (PDA), Obtuse Marginal (OM) and small diagonals. Conditions such as diffuse stenosis and diabetes produce conditions that represent other access and delivery challenges which can be addressed with a delivery system according to the present invention. Other extended treatment areas addressable with the subject systems include vessel bifurcations, chronic total occlusions (CTOs), and prevention procedures (such as in stenting of vulnerable plaque).

Assuming a means of delivering one or more appropriately-sized stents, it may be preferred to use a drug eluting stent in such an application to aid in preventing restenosis. A review of suitable drug coatings and available vendors is presented in “DES Overview: Agents, release mechanism, and stent platform” a presentation by Campbell Rogers, MD incorporated by reference in its entirety. However, bare-metal stents may be employed in the present invention.

While some might argue that the particular role and optimal usage of self expanding stents has yet to be defined, they offer an inherent advantage over balloon expandable stents. The latter type of-devices produce “skid mark” trauma (at least when delivered uncovered upon a balloon) and are associated with a higher risk of end dissection or barotraumas caused at least in part by high balloon pressures and related forces when deforming a balloon-expandable stent for deployment.

Yet, with an appropriate deployment system, self-expanding stents may offer one or more of the following advantages over balloon-expandable models: 1) greater accessibility to distal, tortuous and small vessel anatomy—by virtue of decreasing crossing diameter and increasing compliance relative to a system requiring a deployment balloon, 2) sequentially controlled or “gentle” device deployment, 3) use with low balloon pre-dilatation (if desirable) to reduce barotraumas, 4) strut thickness reduction in some cases reducing the amount of “foreign body” material in a vessel or other body conduit, 5) opportunity to treat neurovasculature—due to smaller crossing diameters and/or gentle delivery options, 6) the ability to easily scale-up a successful treatment system to treat larger vessels or vice versa, 7) a decrease in system complexity, offering potential advantages both in terms of reliability and system cost, 8) reducing intimal hyperplasia, and 9) conforming to tapering anatomy—without imparting complimentary geometry to the stent (though this option exists as well).

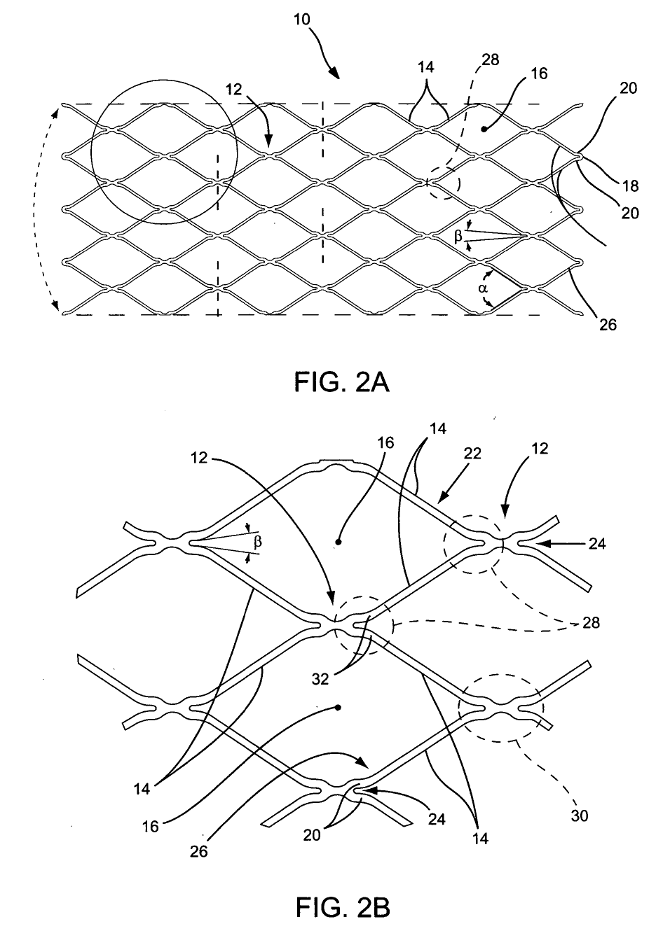

At least some of these noted advantages may be realized using a stent 10 as shown in FIG. 2A. The stent pattern pictured is well suited for use in small vessels. It may be collapsed to an outer diameter of about 0.018 inch (0.46 mm), or even smaller to about 0.014 inch (0.36 mm)—including the restraint/joint used to hold it down—and expand to a size (fully unrestrained) between about 1.5 mm (0.059 inch) or 2 mm (0.079 inch) or 3 mm (0.12 inch) and about 3.5 mm (0.14 inch).

In use, the stent will be sized so that it is not fully expanded when fully deployed against the wall of a vessel in order to provide a measure of radial force thereto (i.e., the stent will be “oversized” as discussed above). The force will secure the stent and offer potential benefits in reducing intimal hyperplasia and vessel collapse or even pinning dissected tissue in apposition.

Stent 10 preferably comprises NiTi that is superelastic at or below room temperature and above (i.e., as in having an A_{f} as low as 15° C. or even 0° C.). Also, the stent is preferably electropolished. The stent may be a drug eluting stent (DES). Such drug can be directly applied to the stent surface(s), or introduced into an appropriate matrix set over at least an outer portion of the stent. It may be coated with gold and/or platinum to provide improved radiopacity for viewing under medical imaging.

In a 0.014 inch delivery system (one in which the maximum nominal outer diameter of the stent/coating and guide member/restraint have a diameter that does not exceed 0.014 inch), the thickness of the NiTi is about 0.0025 inch (0.64 mm) for a stent adapted to expand to 3.5 mm. Such a stent is designed for use in a 3 mm vessel or other body conduit, thereby providing the desired radial force in the manner noted above. Further information regarding radial force parameters in coronary stents may be noted in the article, “Radial Force of Coronary Stents: A Comparative Analysis,” Catheterization and Cardiovascular Interventions 46: 380-391 (1999), incorporated by reference herein in its entirety.

In one manner of production, the stent in FIG. 2A is laser or EDM cut from round NiTi tubing, with the flattened-out pattern shown wrapping around the tube as indicated by dashed lines. In such a procedure, the stent is preferably cut in its fully-expanded shape. By initially producing the stent to full size, the approach allows cutting finer details in comparison to simply cutting a smaller tube with slits and then heat-expanding/annealing it into its final (working) diameter. Avoiding post-cutting heat forming also reduces production cost as well as the above-reference effects.

Regarding the finer details of the subject stent, as readily observed in the detail view provided in FIG. 2B, necked down bridge sections 12 are provided between axially/horizontally adjacent struts or arms/legs 14, wherein the struts define a lattice of closed cells 16. Terminal ends 18 of the cells are preferably rounded-off so as to be atraumatic.

To increase stent conformability to tortuous anatomy, the bridge sections can be strategically separated or opened as indicated by the broken lines in FIG. 2A. To facilitate such tuning of the stent, the bridge sections are sufficiently long so that fully rounded ends 18 may be formed internally to the lattice just as shown on the outside of the stent if the connection(s) is/are severed to separate adjacent cells 16. Whether provided as ends 18 or adjoined by a bridge section 12, junction sections 28 connect circumferentially or vertically adjacent struts (as illustrated). Where no bridge sections are provided, the junction sections can be unified between horizontally adjacent stent struts as indicated in region 30.

The advantage of the optional double-concave profile of each strut bridge 12 is that it reduces material width (relative to what would otherwise be presented by a parallel side profile) to improve flexibility and thus trackability and conformability of the stent within the subject anatomy while still maintaining the option for separating/breaking the cells apart.

Further optional features of stent 10 are employed in the cell end regions 18 of the design. Specifically, strut ends 20 increase in width relative to medial strut portions 22. Such a configuration distributes bending (during collapse of the stent) preferentially toward the mid region of the struts. For a given stent diameter and deflection, longer struts allow for lower stresses within the stent (and, hence, a possibility of higher compression ratios). Shorter struts allow for greater radial force (and concomitant resistance to a radially applied load) upon deployment.

In order to increase stent compliance so that it collapses as much as possible, accommodation is made for the stiffer strut ends 20 provided in the design shown in FIG. 2A. Namely, the gap 24 between the strut ends 22 is set at a smaller angle as if the stent were already partially collapsed in that area. Thus, the smaller amount of angular deflection that occurs at ends 20 can bring the sections parallel (or nearly so) when the strut medial portions 22 are so-arranged. In the variation of the invention in FIG. 2A, radiused or curved sections 26 provide a transition from a medial strut angle α (ranging from about 85 degrees to about 60 degrees) to an end strut angle β (ranging from about 30 to about 0 degrees) at the strut junctions 28 and/or extensions therefrom.

In addition, it is noted that gap 24 an angle β may actually be configured to completely close prior to fully collapsing angle α. The stent shown is not so-configured. Still, the value of doing so would be to limit the strains (and hence, stresses) at the strut ends 22 and cell end regions 18 by providing a physical stop to prevent further strain.

In the detail view of FIG. 2B, angle β is set at 0 degrees. The gap 24 defined thereby by virtue of the noticeably thicker end sections 20 at the junction result in very little flexure along those lever arms. Instead, the strut medial portions accommodate bending. In addition, a hinging effect at the corner or turn 32 of junction section 28 causing rotation of the struts largely about angle α may provide the primary compression mode in this stent.

The stent pattern shown in FIG. 3A and detailed in FIG. 3B offers certain similarities as well as some major differences from that presented in FIGS. 2A and 2B. As in the variation above, stent 40 includes necked down bridge sections 42 provided between adjacent struts or arms/legs 44, wherein the struts define a lattice of closed cells 46. In addition, terminal ends 48 of the cells are preferably rounded-off so as to be atraumatic.

Furthermore, the bridge sections 42 of stent 40 can be separated for compliance purposes. In addition, they may be otherwise modified, such as described above, or even eliminated. Also, in each design, the overall dimensions of the cells and indeed the number of cells provided to define axial length or diameter may be varied (as indicated by the vertical and horizontal section lines in FIG. 3A).

Like the previous stent design, strut ends 50 may offer some increase in width relative to medial strut portions 52. However, as shown in FIG. 3B, as compared to FIG. 2B, the angle β is relatively larger. It is illustrated thus because the overall strut configuration (shape and angles) is not concerned with developing a hinge section and a relatively stiffer outer strut section. Instead, angle β in the FIG. 3A/3B design is meant to collapse and the strut ends are meant to bend in concert with the medial strut portions so as to essentially straighten-out upon collapsing the stent, generally forming tear-drop spaces between adjacent struts—having a stress-reducing radius of curvature where struts join, and compacted to the greatest degree possible at the opposite points to maximize stent compression.

The “S” curves defined by the struts are produced in the stent cut to a final or near final size (as shown in FIGS. 3A and 3B). The curves are preferably determined by virtue of their origination in a physical or computer model that is expanded from a desired compressed shape to the final expanded shape. Thus derived, the stent can be compressed or collapsed under force to provide an outer surface profile that is as solid or smooth and/or cylindrical as possible or feasible.

Such action is enabled by distribution of the stresses associated with compression to generate stains to produce the intended compressed and expanded shapes. This effect is accomplished in a design unaffected by one or more expansion and heat setting cycles that otherwise deteriorate the quality of the superelastic stent material. Further details regarding the “S” stent.

In the case of each of the above stent designs, by utilizing a stent design that minimizes problematic strain (and in the latter case actually uses the same to provide an improved compressed profile), very high compression ratios of the stent may be achieved. Compression ratios (from a fully expanded outside diameter to a fully compressed outside diameter—expressed in those terms used by physicians) of as much as 3.5 mm: 0.014 inch (about 10×) or more are possible—with or without a drug coating and/or restraint used. Compression ratios of 3.0 mm: 0.014 inch (about 8.5×), 3.5 mm: 0.018 inch (about 7.5×), 3.0 mm: 0.018 inch (about 6.5×), 2.5 mm: 0.014 inch (about 7×), 2.5 mm: 0.018 inch (about 5.5×), 2.0 mm: 0.014 inch (about 5.5×), 2.0 mm: 0.018 inch (about 4.5×) offer utility not heretofore possible with existing systems as well.

These selected sizings (and expansion ratios) correspond to treating 1.5 to 3.0 mm vessels by way of delivery systems adapted to pass through existing balloon catheter and microcatheter guidewire lumen. In other words, the 0.014 inch and 0.018 inch systems are designed to corresponding to common guidewire sizes. The system may also be scaled to other common guidewire sizes (e.g., 0.22 inch/0.56 mm or 0.025 inch/0.64 mm) while offering advantages over known systems. Of course, intermediate sizes may be employed as well, especially for full-custom systems. Still further, it is contemplated that the system sizing may be set to correspond to French (FR) sizing. In that case, system sizes contemplated range at least from 1 to 1.5 FR, whereas the smallest known balloon-expandable stent delivery systems are in the size range of about 3 to about 4 FR.

At least when produced at the smallest sizes (whether in an even/standard guidewire or FR size, or otherwise), the system enables a substantially new mode of stent deployment in which delivery is achieved through an angioplasty balloon catheter or small microcatheter lumen. Further discussion and details of “through the lumen” delivery is presented in U.S. patent application Ser. No. 10/746,455 “Balloon Catheter Lumen Based Stent Delivery Systems” filed on Dec. 24, 2003 and its PCT counterpart US2004/008909 filed on Mar. 23, 2004, each incorporated by reference in its entirety.

In “small vessel” cases or applications (where the vessel to be treated has a diameter up to about 3.0 mm), it may also be advantageous to employ a stent delivery system sized at between about 0.022 to about 0.025 inch in diameter. Such a system can be used with catheters compatible with 0.022 inch diameter guidewires.

While such a system may not be suitable for reaching the very smallest vessels, this variation of the invention is quite advantageous in comparison to known systems in reaching the larger of the small vessels (i.e., those having a diameter of about 2.5 mm or larger). By way of comparison, among the smallest known over-the-guidewire delivery systems are the Micro-Driver™ and Pixel™ systems by Guidant. These are adapted to treat vessels between 2 and 2.75 mm, the latter system having a crossing profile of 0.036 inches (0.91 mm). A system described in U.S. Patent Publication No. 2002/0147491 for treating small vessels is purported to be capable of being made as small as 0.026 inch (0.66 mm) in diameter.

With respect to such systems, however, it must be appreciated that a further decrease in stent size may be practically impossible in view of material limitations and functional parameters of the stent. Instead, the present invention offers a different paradigm for delivery devices and stents that are scalable to the sizes noted herein.

By virtue of the approaches taught herein, it is feasible to design system diameters to match (or at least nearly match) common guidewire size diameters (i.e., 0.014, 0.018 and 0.022 inch) for small vessel delivery applications. As noted above, doing so facilitates use of the subject stents with compatible catheters and opens the possibility for methodology employing the same as elaborated upon below and in the above-referenced “Balloon Catheter Lumen Based Stent Delivery Systems” patent application.

Of further note, it may be desired to design a variation of the subject system for use in deploying stents in larger, peripheral vessels, biliary ducts or other hollow body organs. Such applications involve a stent being emplaced in a region having a diameter from about 3.5 to about 13 mm (0.5 inch). In this regard, the scalability of the present system, again, allows for creating a system adapted for such use that is designed around a common wire size. Namely, a 0.035 to 0.039 inch (3 FR) diameter crossing profile system is advantageously provided in which the stent expands (unconstrained) to a size between about roughly 0.5 mm and about 1.0 mm greater than the vessel or hollow body organ to be treated. Sufficient stent expansion is easily achieved with the exemplary stent patterns shown in FIGS. 2A/2B or 3A/3B.

Again, as a matter of comparison, the smallest delivery systems known to applicants for stent delivery in treating such larger-diameter vessels or biliary ducts is a 6 FR system (nominal 0.084 inch outer diameter), which is suited for use in an 8 FR guiding catheter. Thus, even in the larger sizes, the present invention affords opportunities not heretofore possible in achieving delivery systems in the size range of a commonly used guidewire, with the concomitant advantages discussed herein.

Several known stent delivery systems are compatible with (i.e., may be delivered over) common-sized guides wires ranging from 0.014 inch to 0.035 inch (0.89 mm). Yet, none of the delivery systems are themselves known to be so-sized.

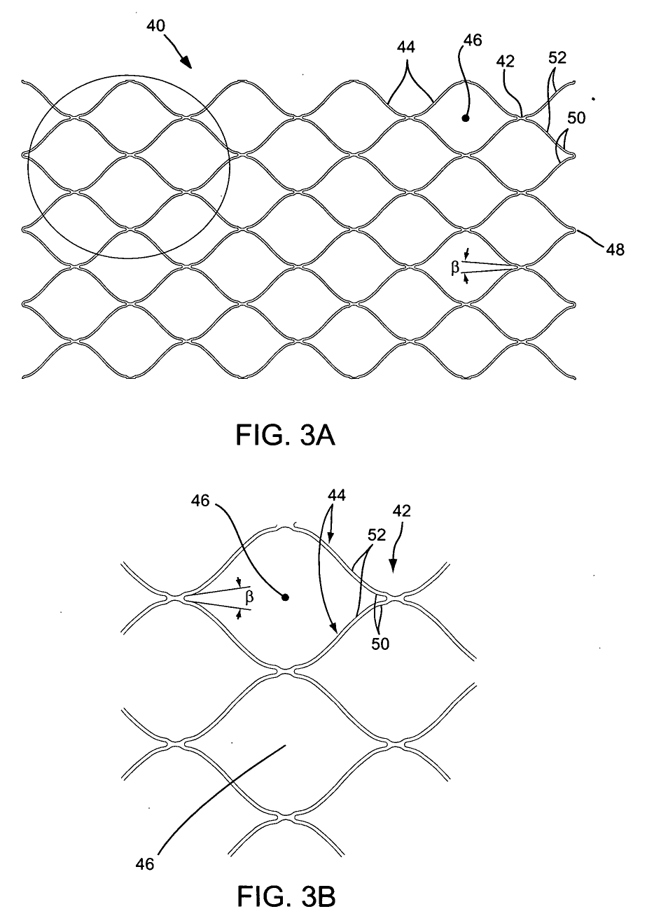

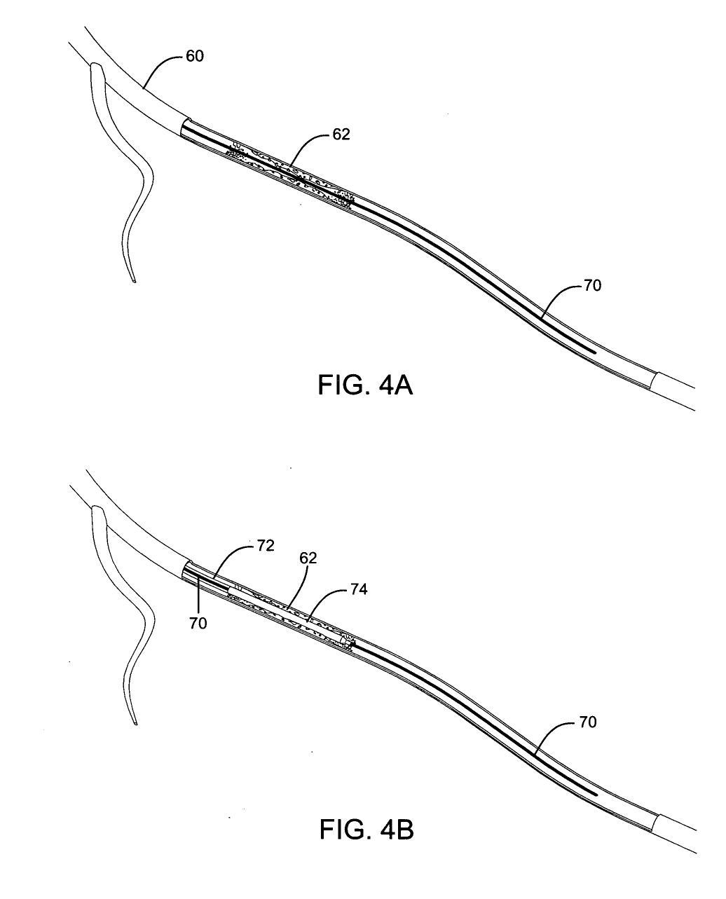

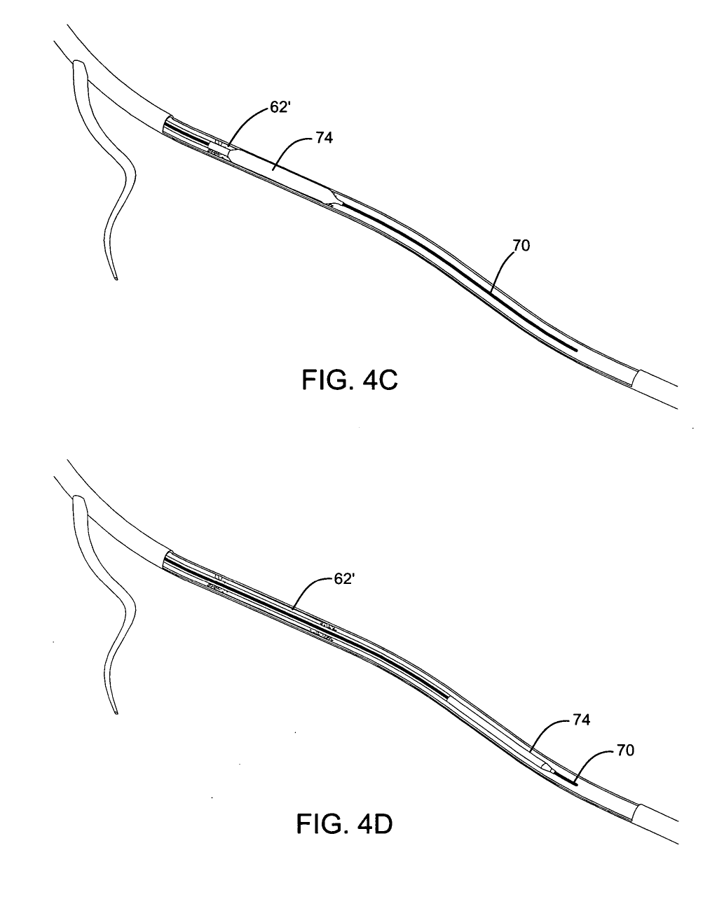

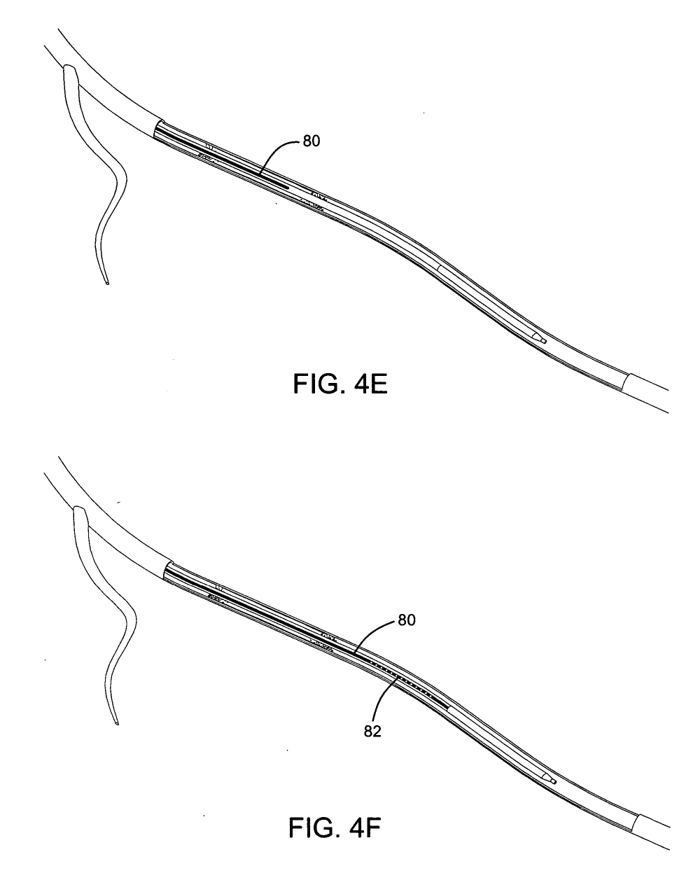

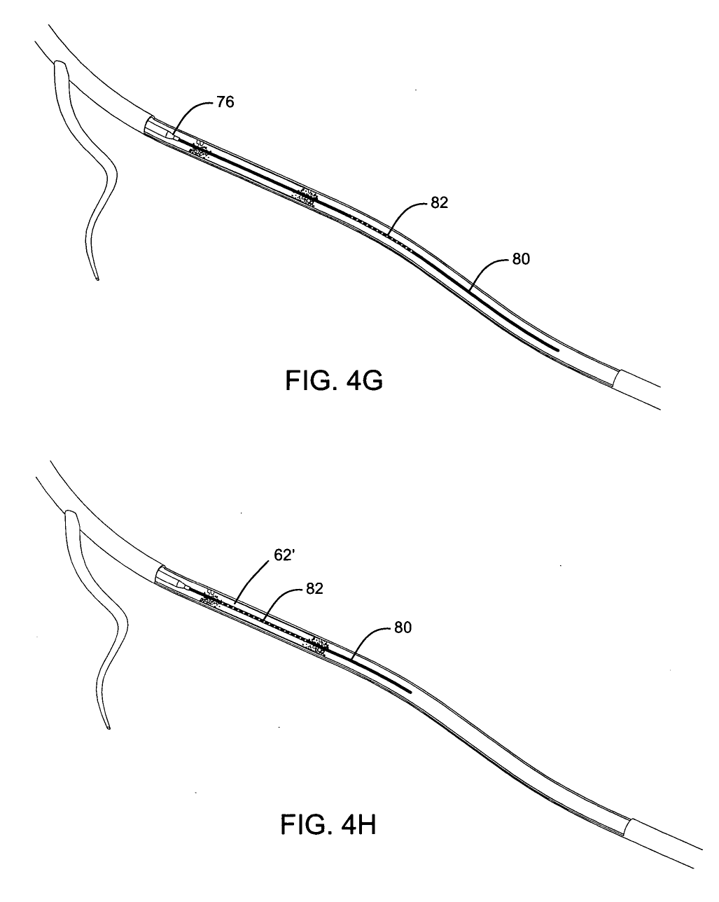

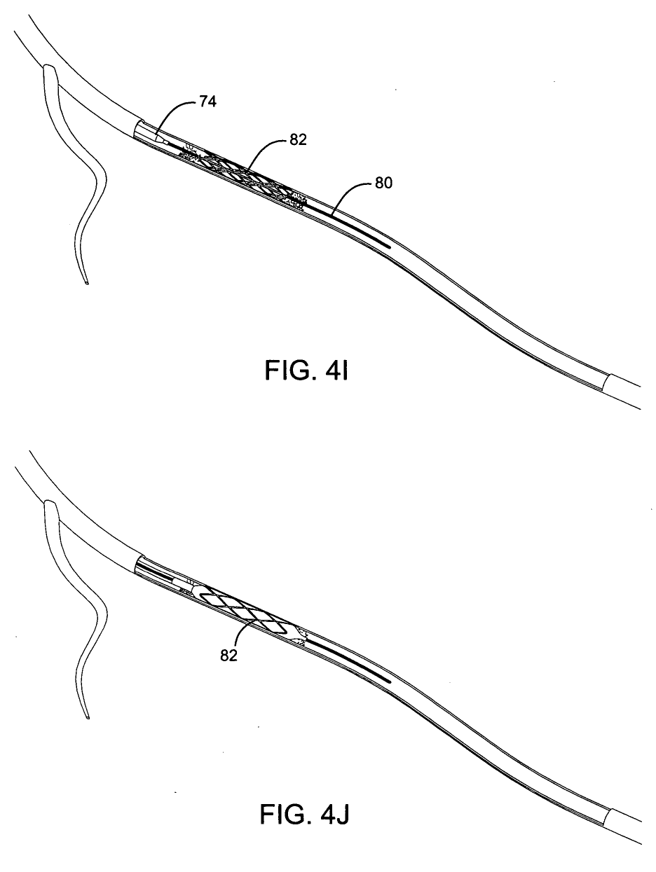

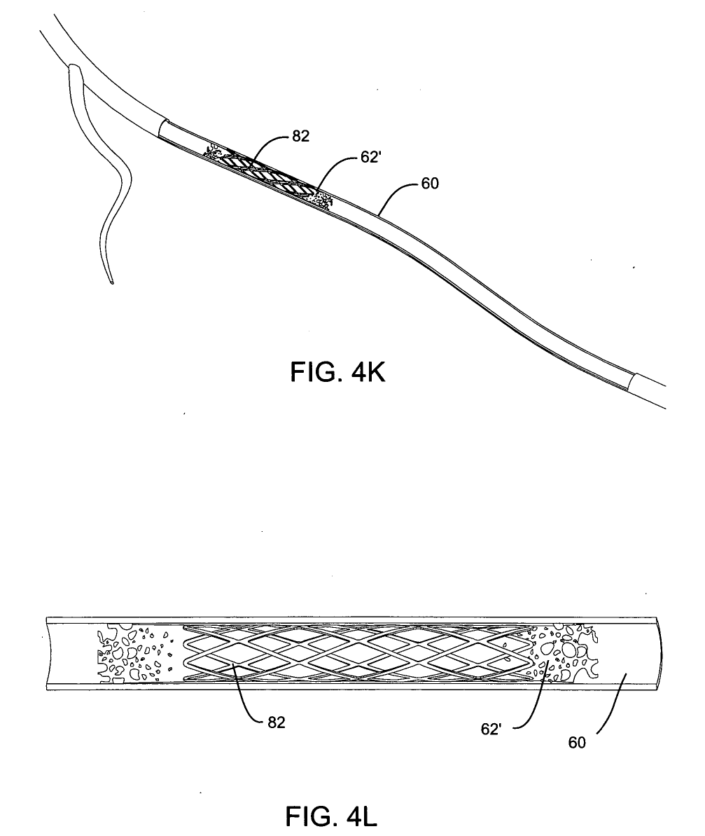

As for the manner of using the inventive system as optionally configured, FIGS. 4A-4L illustrate an exemplary angioplasty procedure. Still, the delivery systems and stents or implants described herein may be used otherwise—especially as specifically referenced herein.

Turning to FIG. 4A, it shows a coronary artery 60 that is partially or totally occluded by plaque at a treatment site/lesion 62. Into this vessel, a guidewire 70 is passed distal to the treatment site. In Fig, 4B, a balloon catheter 72 with a balloon tip 74 is passed over the guidewire, aligning the balloon portion with the lesion (the balloon catheter shaft proximal to the balloon is shown in cross section with guidewire 70 therein).

As illustrated in FIG. 4C, balloon 74 is expanded (dilatated or dialated) in performing an angioplasty procedure, opening the vessel in the region of lesion 62. The balloon expansion may be regarded as “predilatation” in the sense that it will be followed by stent placement (and optionally) a “postdilataton” balloon expansion procedure.

Next, the balloon is at least partially deflated and passed forward, beyond the dilate segment 62′ as shown in FIG. 4D. At this point, guidewire 70 is removed as illustrated in FIG. 4E. It is exchanged for a delivery guide member 80 carrying stent 82 as further described below. This exchange is illustrated in FIGS. 4E and 4F.

However, it should be appreciated that such an exchange need not occur. Rather, the original guidewire device inside the balloon catheter (or any other catheter used) may be that of item 80, instead of the standard guidewire 70 shown in FIG. 4A. Thus, the steps depicted in FIGS. 4E and 4F (hence, the figures also) may be omitted.

In addition, there may be no use in performing the step in FIG. 4D of advancing the balloon catheter past the lesion, since such placement is merely for the purpose of avoiding disturbing the site of the lesion by moving a guidewire past the same. FIG. 4G illustrates the next act in either case. Particularly, the balloon catheter is withdrawn so that its distal end 76 clears the lesion. Preferably, delivery guide 80 is held stationary, in a stable position. After the balloon is pulled back, so is delivery device 80, positioning stent 82 where desired. Note, however, that simultaneous retraction may be undertaken, combining the acts depicted in FIGS. 4G and 4H. Whatever the case, it should also be appreciated that the coordinated movement will typically be achieved by virtue of skilled manipulation by a doctor viewing one or more radiopaque features associated with the stent or delivery system under medical imaging.

Once placement of the stent across from dilated segment 62′ is accomplished, stent deployment commences. The manner of deployment is elaborated upon below. Upon deployment, stent 82 assumes an at least partially expanded shape in apposition to the compressed plaque as shown in FIG. 4I. Next, the aforementioned postdilatation may be effected as shown in FIG. 4J by positioning balloon 74 within stent 82 and expanding both. This procedure may further expand the stent, pushing it into adjacent plaque—helping to secure each.

Naturally, the balloon need not be reintroduced for postdilatation, but it may be preferred. Regardless, once the delivery device 80 and balloon catheter 72 are withdrawn as in FIG. 4K, the angioplasty and stenting procedure at the lesion in vessel 60 is complete. FIG. 4L shows a detailed view of the emplaced stent and the desired resultant product in the form of a supported, open vessel.

In the above description, a 300 cm extendable delivery system is envisioned. Alternatively, the system can be 190 cm to accommodate a rapid exchange of a monorail type of balloon catheter as is commonly known in the art. Of course, other approaches may be employed as well.

Furthermore, other endpoints may be desired such as implanting an anchoring stent in a hollow tubular body organ, closing off an aneurysm, delivering a plurality of stents, etc. In performing any of a variety of these or other procedures, suitable modification will be made in the subject methodology. The procedure shown is depicted merely because it illustrates a preferred mode of practicing the subject invention, despite its potential for broader applicability.

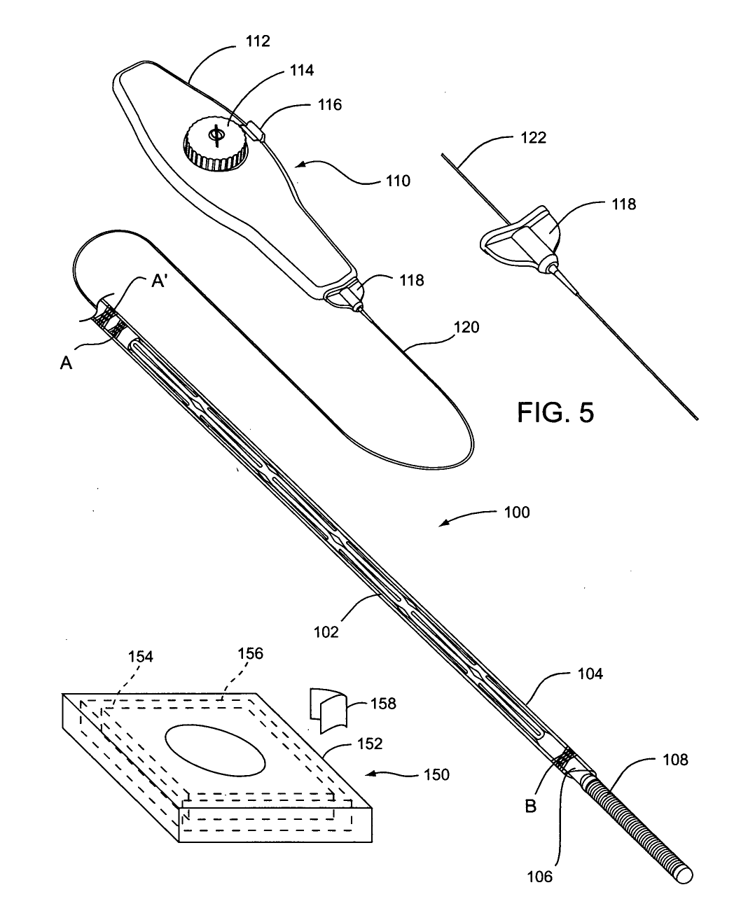

A more detailed overview of the subject delivery systems is provided in FIG. 5. Here, a delivery system 100 is shown along with a stent 102 held in a collapsed configuration upon the delivery guide member. A tubular member 104 is provided over and around the stent to restrain it from expanding. The tubular member may fully surround the stent or only subtend a partial circumference of the stent, it may be split, splittable, comprise a plurality of members or be otherwise provided around the stent to hold or restrain it in a collapsed profile. Tubular member 104 includes a canted or angled distal end 106 presenting a varying axial extent to effect the subject stent release methodology. Further exemplary sheath/restraint end configurations are presented below.

Regarding the overall delivery guide, however, it preferably comprises a flexible atraumatic distal tip 108 of one variety or another. On the other end of the delivery device, a custom handle 110 is preferably provided.

The handle shown is adapted for rotable actuation by holding body 112, and turning wheel 114. It may include a lock 116. Furthermore, a removable interface member 118 facilitates taking the handle off of the delivery system proximal end 120. The interface will be lockable with respect to the body and preferably includes internal features for disengaging the handle from the delivery guide. Once accomplished, it will be possible to attach or “dock” a secondary length of wire 122 on the delivery system proximal end, allowing the combination to serve as an “exchange length” guidewire, thereby facilitating changing-out the balloon catheter or performing another procedure. Alternatively, the wire may be an exchange-length wire.

FIG. 5 also shows packaging 150 containing at least one coiled-up delivery systems 100. When a plurality of such systems are provided (in one package or by way of a number of packages held in stock), they are typically configured in support of a methodology where an appropriate one is picked to reach a target site and deploy a stent without unintended axial movement of the same as per the methodology of Ser. No. 10/792,684, referenced above. Thus, the packaging may serve the purpose of providing a kit or panel of differently configured delivery devices. In the alternative, the packaging may be configured as a tray kit for a single one of the delivery systems.

Either way, packaging may include one or more of an outer box 152 and one or more inner trays 154, 156 with peel-away coverings as is customary in packaging of disposable products provided for operating room use. Naturally, instructions for use 158 can be provided therein. Such instructions may be printed product or be provided in connection with another readable (including computer-readable) medium. The instructions may include provision for basic operation of the subject devices and associated methodology.

Regarding the details of the subject delivery device, it may be provided as in any of the above-referenced patent filings or otherwise, where the sheath or restraint member includes features as further described below. It preferably is one that does not have a section that increases in size during, or after, deployment of the stent. In regard to any delivery system employed, it is to be understood that conventional materials and techniques may be employed in the system construction. In this regard, it will often be desired to provide a lubricious coating or cover between moving components to reduce internal system friction.

In addition, it is to be understood that various radiopaque markers or features may be employed in the system to 1) locate stent position and length, 2) indicate device actuation and stent delivery and/or 3) locate the distal end of the delivery guide. As such, various platinum (or other radiopaque-material) bands or other markers (such as tantalum plugs) may be variously incorporated into the system. Alternatively, or additionally, the stent stop or blocker member may be made of radiopaque material. Especially where the stent employed may shorten somewhat upon deployment, it may also be desired to align radiopaque features with the expected location (relative to the body of the guide member) of the stent upon deployment. For example, it may be desired to incorporate radiopaque features into the restraint and/or bridge or connector sections so that the deployment motion of the device is visible under fluoroscopy. Exemplary markers that may be of use are shown at a proximal end of the stent in FIG. 5 as elements A and A′—on the delivery guide body and tubular member, respectively—and at a distal end of the stent on the restraint as element B.

Regarding more specific aspects of the present invention, FIG. 5 provides a view illustrating the manner in which stent jumping occurs in connection with a simple sheath and pusher type of delivery system as known in the art. Discussion of this known approach is useful in understanding the present invention.

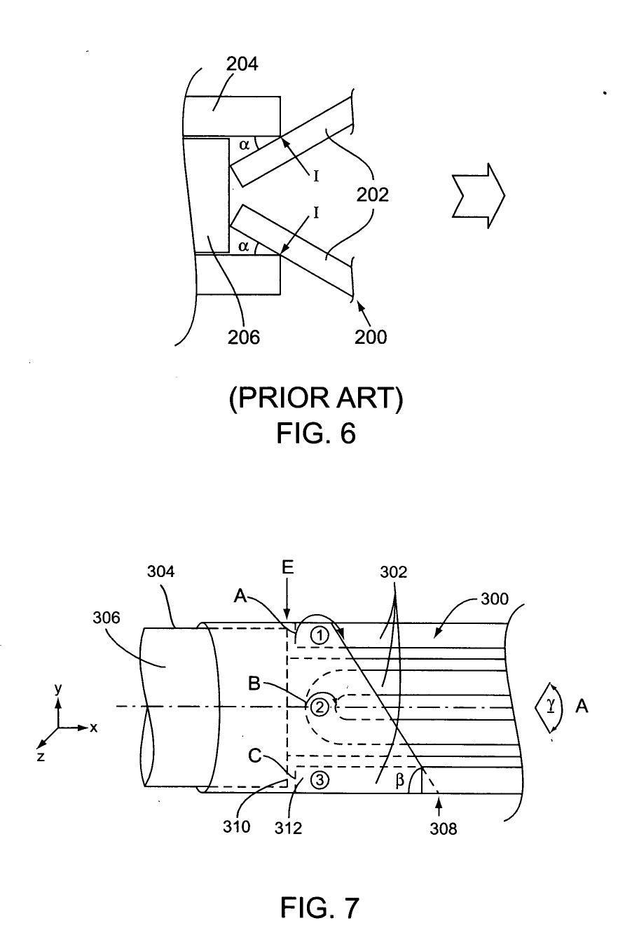

More specifically, FIG. 6 shows a section of a self-expanding stent 200 with its proximal strut ends 202 at a final stage of deployment, in final contact with a sheath 204 and a pusher 206 or abutment feature. Due to the resilient nature of the stent, at a certain point (prior to the pusher aligning with the end of the sheath) the stent will “pop” out of the sheath. The forces generated at interface point “I” due to the angle α between the stent strut ends and the sheath will drive the stent forward as indicated by the arrow.

In contrast, FIG. 7 illustrates an approach of the present invention that alleviates this problem. The figure shows a stent 300 with its proximal end 302 nearly exposed by the delivery system outer tubular sheath or restraint 404 which is partially withdrawn. As easily imagined, further withdrawal of the sheath will cause the struts 302 to exit the tubular member in the order indicated (1), (2), (3) as the terminal points (A), (B), (C) clear the varying axial extent of tubular member 604. Assuming a symmetrical stent arranged as shown, a pair of struts are released at stage (2) as shown in the figure. However, with the stent shown, rotated by an angle γ of 45 degrees relative to tubular member 304 the struts will be released sequentially in two pairs.

In addition to selecting a given alignment for the stent 300 and respective tubular member 304, the stent strut ends 302 need not be aligned as shown. They may vary in their axial extent as well, especially to compliment the action of the restraint by adding greater separation between stages of deployment or additional levels thereof while maintaining a less complex distal configuration for tubular member. Such difference in terminal point location is exemplary indicated in alternate positioning for points (A) and (B), thereby providing greater separation of stage release without altering angle β.

In any case, angle β may be between about 20 and about 80 degrees. An angle lower than 20° may provide an unwieldy system, but is possible. An angle greater than 80° may result in little separation of stent strut release. The end 308 of the tubular member may come to a point when cut on a bias, or be trimmed as shown to avoid any “sharps” in the system.

In addition, as stated previously, the nature of the overall delivery system may vary. In FIG. 7, a basic corewire or inner member 306 is shown, having stop section 310 to abut the proximal strut ends 312 of the stent. Providing such a member to support or abut the interior of the stent helps avoid the angle α type of action noted above in connection with FIG. 6. Also, it will help keep the stent centered or at least located upon the delivery guide, assisting in the stent strut release approaches discussed below in which multiple ones of the struts are released in an asymmetrical fashion.

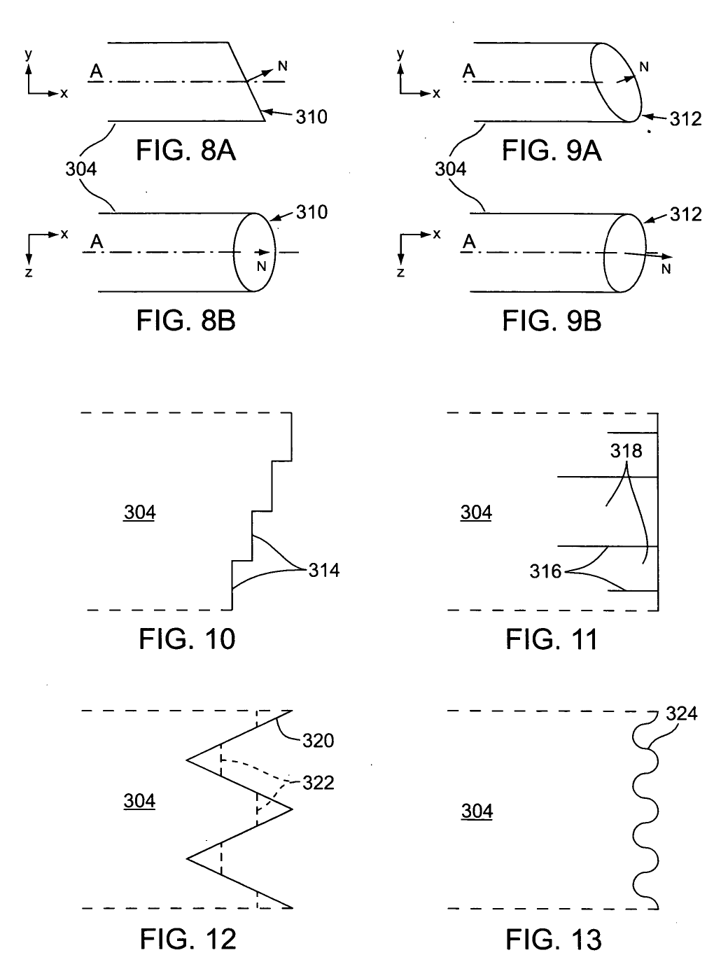

The tubular restraint shown in FIG. 7 is detailed in FIGS. 8A and 8B. Here, it is made clear that a distal opening 310 to the restraint is elliptical. The same is true for opening 312 of the tubular restraint member in FIGS. 9A and 9B.

As illustrated in both FIGS. 8A/8B and 9A/9B, the plane of each ellipse has normal axis “N” thereto that is offset, canted or skewed relative to an axis “A” of the tubular member body.

In the approach shown in FIGS. 8A and 8B, normal axis N is aligned with axis A when viewed along the “Y” axis shown. It is otherwise set askew or in an asymmetrical fashion relative to these axes. In the approach in FIGS. 9A and 9B, the normal axis N offers no such alignment. Without this alignment and a stent having struts that terminate at the same axial extent “E” along axis “A” as shown in FIG. 7, the varying axial extent of opening 352 results in individual release of the stent ends. In contrast, for the approach shown in FIGS. 8A and 8B, where the stent is oriented as shown in FIG. 7 the release will be as described above.

FIGS. 10-13 show expanded cut patterns as may be used in producing other tubular stent-restraining members according to the present invention. Variation 304 shown in FIG. 10 shows multiple sequential steps 314; variation 304 in FIG. 11 is a slit/flapped approach where the varying-length slits 316 define different size flaps 318 that will have different resistance to opening thereby releasing the stent strut ends differentially while still offering a closed-off system; variation 304 in FIG. 12 has a zig-zag 320 or cropped zig-zag pattern (as indicated by phantom line 322); and variation 304 in FIG. 12 varies in axial extent via a sinusoidal end pattern 324.

As will be readily appreciated upon review of the figures, the tubular members shown allow for the staged fashion of deployment. The particular end configurations shown allow for stepwise stent end release in a variety of manners. Depending at least upon the rotational orientation of the stent, these exemplary approaches include situations in which: 1) at least some of the proximal strut ends are individually released—FIGS. 8A/8B, 9A/9B and 10-13; 2) all of the proximal strut ends are individually released—FIGS. 9A/9B, 10 and 11; 3) more than two adjacent ones of the proximal strut ends are released sequentially—FIGS. 8A/8B, 9A/9B, 10 and 11; 4) at least some of the proximal strut ends are released in a symmetrical fashion—FIGS. 8A/8B, 12 and 13; 5) opposing pairs of the proximal strut ends are simultaneously released one pair after the other—FIGS. 8A/8B and 12; 6) higher multiple sets of proximal stent ends are released after each other—FIG. 13; and 7) only one proximal strut end is held prior to completing stent deployment—FIGS. 8A/8B, 9A/9B and 10-13. Other options as alluded to above or as may be appreciated by those with skill in the art exist as well.

Though the invention has been described in reference to several examples, optionally incorporating various features, the invention is not to be limited to that which is described or indicated as contemplated with respect to each embodiment or variation of the invention. The breadth of the present invention is to be limited only to the broadest possible scope of the following claims, in which the claims are interpreted given the plain meaning of terms, modified only to account for any explicit definition herein or as relied upon during prosecution. In this context, we claim:

1. A stent delivery system comprising:
a self-expanding stent having a plurality of proximal termination points, and
a delivery guide comprising a tubular member restraining the stent in a collapsed configuration, the tubular member having a distal end with a varying axial extent,
wherein withdrawal of the tubular member releases the stent such that the varying axial extent releases some of a plurality of the proximal terminal points in a step-wise fashion to alleviate stent jumping upon delivery.

2. The system of claim 1, wherein the varying axial extent is configured so that at least some of the proximal terminal points are individually released.

3. The system of claim 2, wherein the varying axial extent is configured so that all of the proximal terminal points are individually released.

4. The system of claim 2, wherein the varying axial extent is configured so that adjacent ones of the proximal terminal points are released sequentially.

5. The system of claim 1, wherein the varying axial extent is configured so that at least some of the proximal termination points are released in a symmetrical fashion.

6. The system of claim 5, wherein the varying axial extent is configured so that opposing pairs of the proximal terminations points are simultaneously released.

7. The system of claim 1, wherein the varying axial extent is configured so that less than half of the proximal termination points are restrained within the tubular member prior to completing stent deployment.

8. The system of claim 7, wherein the varying axial extent is configured so that only one proximal termination point is restrained within the tubular member prior to completing stent deployment.

9. The system of claim 1, wherein the tubular member is cylindrical and has an elliptical distal opening.

10. The system of claim 1, wherein a distal opening of the tubular member is flapped.

11. The system of claim 1, wherein a distal opening of the tubular member is stepped.

12. The system of claim 1, wherein less than half of the proximal strut ends are released to effect final release of the stent.

13. The system of claim 1, wherein the proximal terminal points are aligned axially with a delivery guide axis, whereby only the varying axial extent of the tubular member is configured so effect the step-wise release.

14. A stent delivery guide comprising:
a tubular member adapted to restrain a stent in a collapsed configuration, the tubular member having a distal end with a varying axial extent.

15. The stent delivery guide of claim 14, further comprising an inner member adapted to support an interior surface of the stent.

16. The stent delivery guide of claim 14, wherein the delivery guide is adapted to carry only one stent.

17. The stent delivery system of claim 14, wherein the distal end has an angled tip set at between about 20 and about 80 degrees relative to an axis of the delivery guide.

18. A method of stent delivery, the method comprising:
positioning a stent at a target site, the stent having a plurality of proximal terminal points, and
first releasing more than half of the proximal terminal points, then releasing the remaining terminal points to complete stent release.

19. A method of stent delivery, the method comprising:
positioning a stent at a target site, the stent having a plurality of proximal terminal points axially aligned with a delivery guide member, and
first releasing at least some of the proximal terminal points, then releasing the remaining terminal points to complete stent release.

20. The method of claim 18, wherein more than half of the proximal terminal points are first released.
